(19) United States
(12) Patent Application Publication
(10) Pub. No.: US 2025/0095801 A1
(43) Pub. Date: Mar. 20, 2025

(54) INFORMATION PROCESSING SYSTEM AND INFORMATION PROCESSING METHOD

(71) Applicant: SHIMADZU CORPORATION, Kyoto-shi (JP)

(72) Inventors: Yusuke MURASE, Kyoto-shi (JP); Sachiko KOBAYASHI, Kyoto-shi (JP); Osamu MOTOKAWA, Kyoto-shi (JP); Masaki KANAI, Kyoto-shi (JP)

(21) Appl. No.: 18/294,939

(22) PCT Filed: Aug. 5, 2021
(86) PCT No.: PCT/JP2021/029048

§ 371 Date: Apr. 8, 2024

(51) Int. Cl. G16H 10/60; G16H 40/67; G16H 80/00

(52) U.S. Cl. CPC G16H 10/60; G16H 40/67; G16H 80/00

(57) ABSTRACT

An information processing system includes: a measuring device that measures advanced glycation end products (AGEs) of a user and is provided with identification information of a supporter who supports the user; and a server device that communicates with the measuring device and a supporter terminal of the supporter. The measuring device outputs a measurement result of the AGEs in association with the identification information to the server device. The server device stores the measurement result in association with the identification information and outputs information about the measurement result to the supporter terminal.

### TECHNICAL FIELD

The present disclosure relates to an information processing system and an information processing method.

### BACKGROUND ART

One of the substances that cause aging is advanced glycation end products (hereinafter also referred to as “AGEs”). The AGEs are a generic term for a plurality of compounds and are generated by saccharides and proteins binding to each other and reacting by oxidation, dehydration and condensation. It is believed that the AGEs are accumulated in the body due to disordered lifestyle habits, and cause age-related diseases and lifestyle-related diseases (e.g., diabetes and dementia).

A measuring device that measures AGEs has been conventionally known. For example, PTL 1 discloses a small-sized sensor that receives fluorescence excited by light applied to the skin of a user and measures a degree of accumulation of AGEs based on the intensity of the received fluorescence.

### CITATION LIST

### Patent Literature

PTL 1: Japanese Patent Laying-Open No. 2015-223431

### SUMMARY OF INVENTION

### Technical Problem

By using the sensor disclosed in PTL 1, a user can easily measure AGEs. The sensor that measures the AGEs is often placed in a pharmacy, a medical institution, a nursing care facility, a gym and the like. The user typically measures the AGEs when visiting these facilities on business. When the user desires a closer examination, the user can take a blood test in a medical institution to measure the AGEs.

A user who is a subject of measurement of AGEs may in some cases be supported by a supporter. When a user who needs to undergo measurement of AGEs is an older person, a supporter may be a staff member of a nursing care facility. The supporter supports the user in consideration of a measurement result of the AGEs, a body measurement result such as a walking speed, a body weight, the number of steps, a body mass index (hereinafter also referred to as “BMI”), a skeletal muscle mass index (hereinafter also referred to as “SMI”), and a grip strength, a daily medical consultation result, a mild cognitive impairment (hereinafter also referred to as “MCI”) test result, and a functional training result. The user reconsiders his/her lifestyle habits in accordance with the support provided by the supporter, thereby improving the AGEs.

In view of the above-described circumstances, not only a user and a user's family but also a supporter and a nursing care facility manager need to know a measurement result of AGEs obtained by the user. Thus, there is a need for a mechanism that allows a user and a supporter to conveniently share a measurement result of AGEs. Such sharing of the measurement result of the AGEs is also required in an exercise facility, a dispensing pharmacy, a drug store, hairdressing business, a convenience store, a supermarket, a health promotion-related department in each prefecture, and a company's health insurance office.

The present disclosure has been made to solve the above-described problem, and an object thereof is to provide the technique of conveniently sharing a measurement result of AGEs.

### Solution to Problem

An information processing system according to an aspect of the present disclosure includes: a measuring device that measures advanced glycation end products (AGEs) of a user and is provided with identification information of a supporter who supports the user; and a server device that communicates with the measuring device and a supporter terminal of the supporter. The measuring device outputs a measurement result of the AGEs in association with the identification information to the server device. The server device stores the measurement result in association with the identification information and outputs information about the measurement result to the supporter terminal.

An information processing method according to another aspect of the present disclosure includes: (a) outputting, by a measuring device, to a server device, a measurement result of AGEs of a user in association with identification information of a supporter who supports the user; (b) storing, by the server device, the measurement result in association with the identification information; and (c) outputting, by the server device, information about the measurement result to a supporter terminal of the supporter.

### Advantageous Effects of Invention

According to the present disclosure, a measurement result of AGEs can be conveniently shared.

### BRIEF DESCRIPTION OF DRAWINGS

FIG. 1 shows an information processing system according to a first embodiment.

FIG. 2 shows a configuration of a server device according to the first embodiment.

FIG. 3 is a diagram for illustrating a user identification information table stored in the server device according to the first embodiment.

FIG. 4 is a diagram for illustrating a supporter identification information table stored in the server device according to the first embodiment.

FIG. 5 is a diagram for illustrating a device identification information table stored in the server device according to the first embodiment.

FIG. 6 is a diagram for illustrating a viewing information table stored in the server device according to the first embodiment.

FIG. 7 shows an example of a display screen in a user terminal, a supporter terminal or a viewer terminal according to the first embodiment.

FIG. 8 shows an example of a display screen in the user terminal, the supporter terminal or the viewer terminal according to the first embodiment.

FIG. 9 shows an example of a display screen in the user terminal, the supporter terminal or the viewer terminal according to the first embodiment.

FIG. 10 shows an example of a display screen in the user terminal, the supporter terminal or the viewer terminal according to the first embodiment.

FIG. 11 shows an example of a display screen in the user terminal, the supporter terminal or the viewer terminal according to the first embodiment.

FIG. 12 shows an example of a display screen in the user terminal, the supporter terminal or the viewer terminal according to the first embodiment.

FIG. 13 shows an example of a display screen in the user terminal, the supporter terminal or the viewer terminal according to the first embodiment.

FIG. 14 is a flowchart of a storage process performed by the information processing system according to the first embodiment.

FIG. 15 is a flowchart of a viewing process performed by the information processing system according to the first embodiment.

FIG. 16 shows a modification of the information processing system according to the first embodiment.

FIG. 17 shows an information processing system according to a second embodiment.

FIG. 18 shows a modification of the information processing system according to the second embodiment.

### DESCRIPTION OF EMBODIMENTS

The present embodiment will be described in detail with reference to the drawings, in which the same or corresponding portions are denoted by the same reference characters and description thereof will not be repeated in principle.

### <Information Processing System According to First Embodiment>

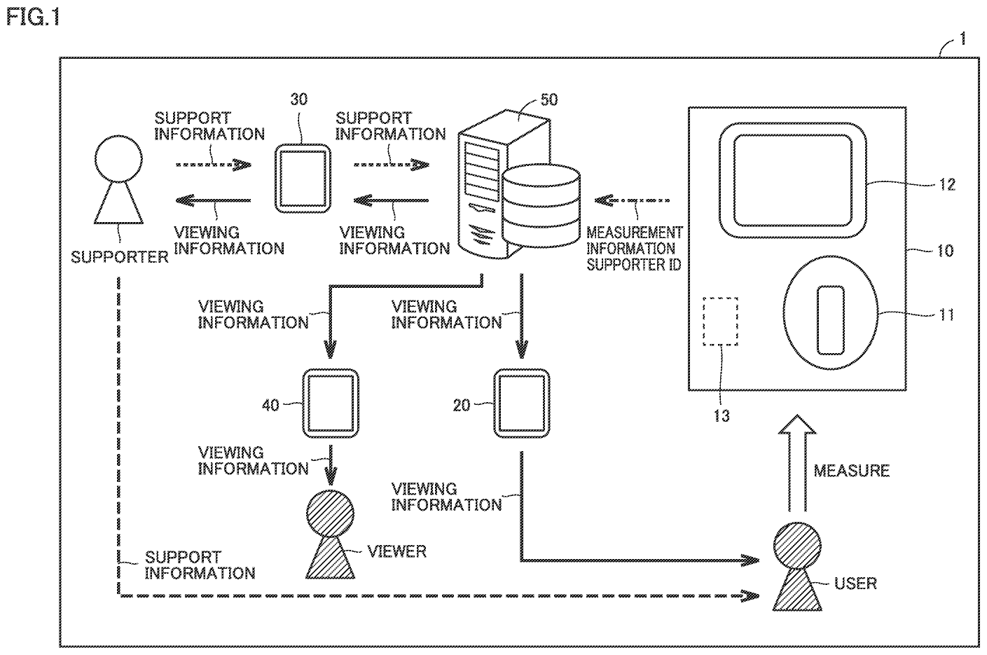

An information processing system 1 according to a first embodiment will be described with reference to FIGS. 1 to 16.

### [Configuration of Information Processing System]

FIG. 1 shows information processing system 1 according to the first embodiment. As shown in FIG. 1, information processing system 1 includes a measuring device 10, a user terminal 20, a supporter terminal 30, a viewer terminal 40, and a server device 50.

Measuring device 10 is a device for measuring AGEs of a user. Measuring device 10 includes a measuring unit 11, a display 12 and a communication unit 13. Although measuring device 10 includes display 12 in the present embodiment, display 12 may be omitted. Measuring unit 11 measures the AGEs of the user in a non-invasive manner. Some of a plurality of compounds included in the AGEs have the property of emitting fluorescence by being irradiated with specific light. Measuring unit 11 uses such property of the compounds to measure the AGEs of the user.

When the user causes his/her fingertip to touch measuring unit 11, measuring unit 11 applies light to the skin from a not-shown light source. Measuring unit 11 may apply light to the skin of a body part (e.g., arm) other than the fingertip of the user. The light applied by measuring unit 11 is, for example, excitation light having a peak in a wavelength range of equal to or less than 410 nm. Measuring unit 11 receives fluorescence excited by the light applied to the skin at a not-shown light receiving element, and measures a degree of accumulation of the AGEs based on the intensity of the received fluorescence. Display 12 displays a measurement result of the AGEs obtained by measuring unit 11. The measurement result includes, for example, the intensity of the fluorescence received by measuring unit 11, and a value of the degree of accumulation of the AGEs converted into a score. The measurement result may include the intensity of the fluorescence received by measuring unit 11, and a corrected value obtained by correcting the value of the degree of accumulation of the AGEs converted into a score.

Communication unit 13 receives and transmits data (information) to and from server device 50 by wired communication or wireless communication. Communication unit 13 is an information terminal that can communicate with server device 50 through the network, such as a desktop personal computer (PC), a laptop PC, a smartphone, a smart watch, a wearable device, a tablet PC, and a network adapter. Communication unit 13 may be integrated with display 12. Communication unit 13 may be separate from each of measuring unit 11 and display 12.

Measuring device 10 is placed in various facilities such as a pharmacy, a medical institution, a nursing care facility, and a gym. Measuring device 10 is managed by a supporter. For example, the supporter rents measuring device 10 from a manufacturer of measuring device 10 and identification information (supporter ID) of the supporter is assigned to measuring device 10. When the supporter measures the AGEs of the user supported by the supporter himself/herself by using measuring device 10, a measurement result of the AGEs is transmitted from measuring device 10 to server device 50 together with the supporter ID.

User terminal 20 is owned or used by the user. User terminal 20 is an information terminal that can communicate with server device 50 through the network, such as, for example, a desktop PC, a laptop PC, a smartphone, a smart watch, a wearable device, and a tablet PC. By directly or indirectly accessing server device 50 using user terminal 20, the user can obtain information about the measurement result of the AGEs of the user stored in server device 50.

Supporter terminal 30 is owned or used by the supporter who supports the user. Supporter terminal 30 is an information terminal that can communicate with server device 50 through the network, such as, for example, a desktop PC, a laptop PC, a smartphone, a smart watch, a wearable device, and a tablet PC. By directly or indirectly accessing server device 50 using supporter terminal 30, the supporter can obtain the information about the measurement result of the AGEs of the user stored in server device 50.

The user is a person that uses service (hereinafter also referred to as “information providing service”) provided by information processing system 1. More specifically, the user includes an older person that uses a nursing care facility, and a person having age-related diseases and lifestyle-related diseases. The user is a target of measurement (subject of measurement) of the AGEs using measuring device 10.

The supporter is a person that uses the information providing service, similarly to the user, and is a person or an organization that supports the user. As one example, the supporter may be a staff member, a nurse or a lifestyle counselor in a day-to-day-type nursing care facility (elderly day care center or rehabilitation day care center) or a nursing care facility. As another example, the supporter may be an instructor or a nutrition adviser in a fitness gym. Although there are individual differences, a measurement value of the AGEs generally changes in one to several weeks. Even when the supporter does not communicate with the user every day (e.g., even when the frequency of the communication is approximately once a week), the supporter can obtain the change in the AGEs by measuring the AGEs with this frequency. Although a human is illustrated as an example of the supporter in the present embodiment, the supporter may be a machine (e.g., a robot) having artificial intelligence mounted thereon. In this case, the supporter terminal used by the supporter may be the machine itself having artificial intelligence mounted thereon.

Viewer terminal 40 is owned or used by a viewer. Viewer terminal 40 is an information terminal that can communicate with server device 50 through the network, such as, for example, a desktop PC, a laptop PC, a smartphone, a smart watch, a wearable device, and a tablet PC. By directly or indirectly accessing server device 50 using viewer terminal 40, the viewer can obtain the information about the measurement result of the AGEs of the user stored in server device 50.

The viewer is a family or a relative of the user, or a relevant person (e.g., an acquaintance) related to the user, who is authorized to view the measurement result about the user by the user or the supporter.

Server device 50 is managed by a service provider who provides the information providing service. The service provider may be the manufacturer of measuring device 10 that rents measuring device 10 to the supporter. Server device 50 functions as a cloud computer, thereby communicating with each of measuring device 10, user terminal 20 and supporter terminal 30.

In information processing system 1 configured as described above, when the user measures the AGEs using measuring device 10, measuring device 10 outputs the measurement result of the AGEs in association with the supporter ID to server device 50. Server device 50 obtains measurement information including the measurement result from measuring device 10 and stores the measurement information in association with the supporter ID. Server device 50 accumulates and stores the past measurement information of the AGEs obtained by the user using measuring device 10.

Furthermore, the supporter inputs support information to supporter terminal 30 in consideration of the measurement result of the AGEs of the user obtained from server device 50. The support information is information for supporting the user and information about lifestyle-related diseases prevention of the user. The support information includes, for example, other measurement results of the user other than the AGEs, and nursing care information that is valuable for the user. The nursing care information includes, for example, an advice about dementia prevention. The other measurement results include, for example, a body measurement result of the user such as a walking speed, a body weight, the number of steps, a BMI, and a grip strength, a daily medical consultation result, an MCI test result, and a functional training result. Supporter terminal 30 outputs the support information to server device 50.

Server device 50 obtains the support information from supporter terminal 30 and stores the support information. The supporter may directly notify the user about the support information without the intervention of supporter terminal 30 and server device 50.

Based on the measurement information obtained from measuring device 10 and stored, server device 50 generates viewing information to be viewed by each of the user, the supporter and the viewer. In addition to the measurement information, server device 50 may generate the viewing information based on the support information obtained from supporter terminal 30. Furthermore, server device 50 may analyze a health condition of the user based on the measurement information and the support information, and generate analysis information including an analysis result. In other words, the viewing information includes the measurement information. The viewing information may further include at least one of the support information and the analysis information, in addition to the measurement information.

When the user requests the viewing information using user terminal 20, server device 50 outputs the viewing information to user terminal 20 in response to the request from user terminal 20. User terminal 20 displays the viewing information obtained from server device 50.

As a result, the user can view the measurement information of the AGEs accumulated and stored in server device 50, using user terminal 20. When the viewing information includes the support information, the user can view the support information stored in server device 50, using user terminal 20. Furthermore, when the viewing information includes the analysis information, the user can view the analysis information stored in server device 50, using user terminal 20.

When the supporter requests the viewing information including the measurement information using supporter terminal 30, server device 50 outputs the viewing information to supporter terminal 30 in response to the request from supporter terminal 30. Supporter terminal 30 displays the viewing information obtained from server device 50.

As a result, the supporter can view the measurement information of the AGEs of the user accumulated and stored in server device 50, using supporter terminal 30. When the viewing information includes the support information, the supporter can view the support information stored in server device 50, using supporter terminal 30. Furthermore, when the viewing information includes the analysis information, the supporter can view the analysis information stored in server device 50, using supporter terminal 30. Particularly, in the present embodiment, the supporter can obtain the viewing information associated with his/her own supporter ID from server device 50 using supporter terminal 30. Thus, the supporter can obtain, from server device 50, measurement results of the AGEs measured by measuring device 10 managed by the supporter himself/herself, i.e., measurement results of the AGEs of all of the users supported by the supporter himself/herself, and view the measurement results of the AGEs.

When the viewer requests the viewing information using viewer terminal 40, server device 50 outputs the viewing information to viewer terminal 40 in response to the request from viewer terminal 40. Viewer terminal 40 displays the viewing information obtained from server device 50.

As a result, the viewer can view the measurement information of the AGEs accumulated and stored in server device 50, using viewer terminal 40. When the viewing information includes the support information, the viewer can view the support information stored in server device 50, using viewer terminal 40. Furthermore, when the viewing information includes the analysis information, the viewer can view the analysis information stored in server device 50, using viewer terminal 40.

### [Configuration of Server Device]

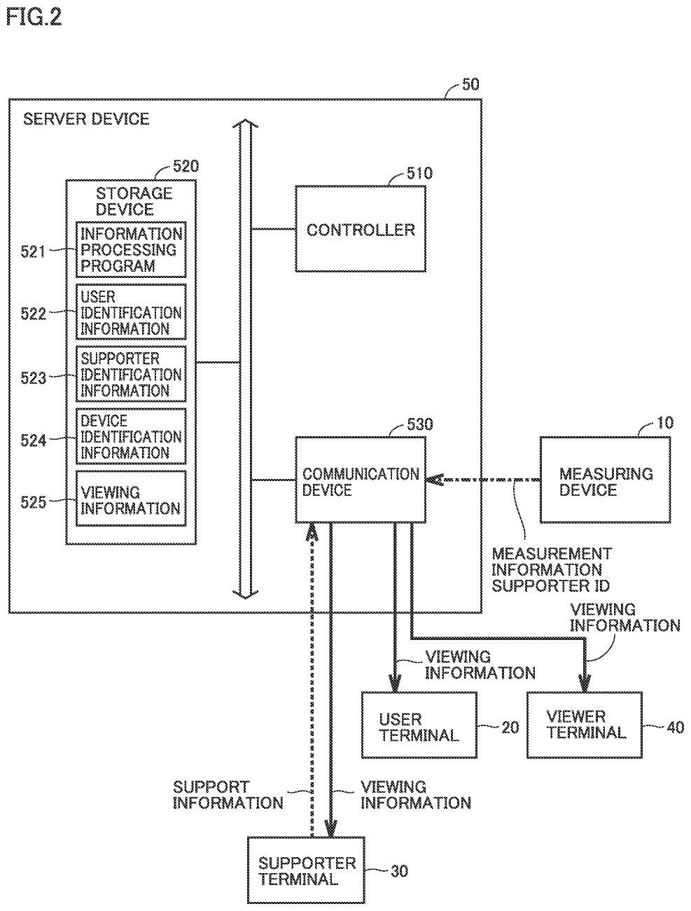

FIG. 2 shows a configuration of server device 50 according to a second embodiment. As shown in FIG. 2, server device 50 includes a controller 510, a storage device 520 and a communication device 530.

Controller 510 is an example of a computer and is a computing entity that performs various types of processing in accordance with various programs. Controller 510 includes, for example, at least one of a central processing unit (CPU), a field programmable gate array (FPGA), a graphics processing unit (GPU), and a multi processing unit (MPU). Controller 510 may further include a volatile memory such as a dynamic random access memory (DRAM) and a static random access memory (SRAM) and/or a non-volatile memory such as a read only memory (ROM) and a flash memory. Controller 510 may be implemented by processing circuitry.

Storage device 520 includes a non-volatile memory such as a hard disk drive (HDD) and a solid state drive (SSD). Storage device 520 stores various types of programs and data such as an information processing program 521 performed by controller 510, and user identification information 522, supporter identification information 523, device identification information 524, and viewing information 525 to which controller 510 refers.

User identification information 522 includes information about the user, such as a user ID, a password, a user name, and an affiliation. Server device 50 can identify the user using user identification information 522.

Supporter identification information 523 includes information about the supporter, such as a supporter ID, a password, a supporter name, and an affiliation. Server device 50 can identify the supporter using supporter identification information 523.

Device identification information 524 includes information about measuring device 10, such as a device ID, a model number, a serial number, and a location of placement. Server device 50 can identify measuring device 10 using device identification information 524.

Viewing information 525 includes the measurement information including the measurement result of the AGEs of the user. Viewing information 525 may further include at least one of the support information provided from the supporter and the analysis information including the analysis result based on the measurement information and the support information.

Storage device 520 may include a volatile memory such as a DRAM and an SRAM and/or a non-volatile memory such as a ROM and a flash memory. Controller 510 may further include a not-shown media reader. Controller 510 may receive, through the media reader, a removable disk serving as a storage medium, and obtain various types of programs and data such as information processing program 521, user identification information 522, supporter identification information 523, and device identification information 524 from the removable disk.

Communication device 530 receives and transmits data (information) to and from each of measuring device 10, user terminal 20 and supporter terminal 30 by wired communication or wireless communication.

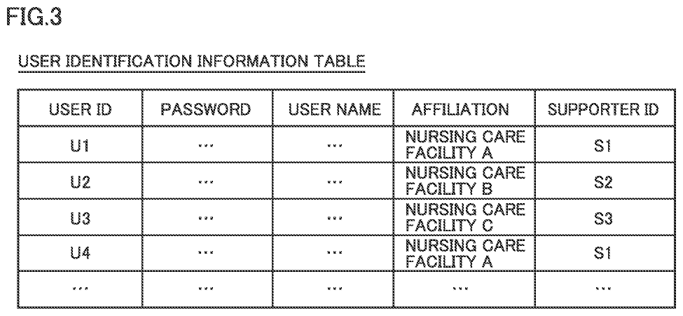

FIG. 3 is a diagram for illustrating a user identification information table stored in server device 50 according to the first embodiment. Server device 50 stores user identification information 522 using the user identification information table shown in FIG. 3.

As shown in FIG. 3, the user identification information table stores information about a user ID, a password, a user name, an affiliation and the like as user identification information 522. Each user who uses the information providing service is identified by user identification information 522. For example, “U1” is assigned to a first user as the user ID, and “U2” is assigned to a second user as the user ID. In the user identification information table, each user ID is associated with a supporter ID of a supporter who supports the user to which this user ID is assigned. For example, “S1” is associated with the user of “U1” as the supporter ID, and “S2” is associated with the user of “U2” as the supporter ID.

The password, the user name and the affiliation, of user identification information 522, are input to user terminal 20 by each user. User terminal 20 outputs, to server device 50, user identification information 522 input to user terminal 20. Server device 50 stores user identification information 522 obtained from user terminal 20 in the user identification information table, thereby storing user identification information 522 in storage device 520.

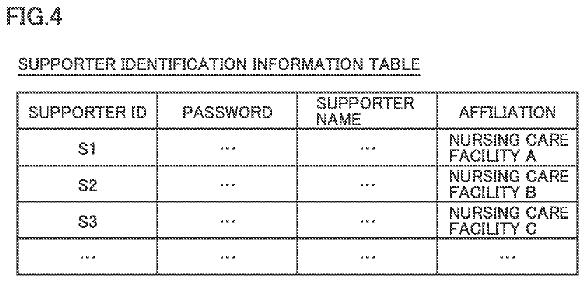

FIG. 4 is a diagram for illustrating a supporter identification information table stored in server device 50 according to the first embodiment. Server device 50 stores supporter identification information 523 using the supporter identification information table shown in FIG. 4.

As shown in FIG. 4, the supporter identification information table stores information about a supporter ID, a password, a supporter name, an affiliation and the like as supporter identification information 523. Each supporter who uses the information providing service is identified by supporter identification information 523. For example, “S1” is assigned to a first supporter as the supporter ID, and “S2” is assigned to a second supporter as the supporter ID.

The password, the user name and the affiliation, of supporter identification information 523, are input to supporter terminal 30 by each supporter. Supporter terminal 30 outputs, to server device 50, supporter identification information 523 input to supporter terminal 30. Server device 50 stores supporter identification information 523 obtained from supporter terminal 30 in the supporter identification information table, thereby storing supporter identification information 523 in storage device 520.

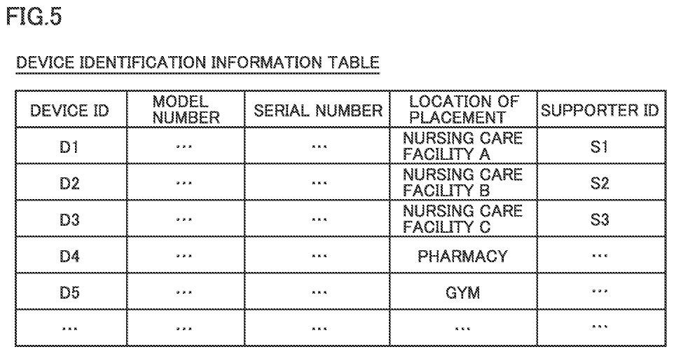

FIG. 5 is a diagram for illustrating a device identification information table stored in server device 50 according to the first embodiment. Server device 50 stores device identification information 524 using the device identification information table shown in FIG. 5.

As shown in FIG. 5, the device identification information table stores information about a device ID, a model number, a serial number, a location of placement and the like as device identification information 524. Each measuring device 10 is identified by device identification information 524. For example, “D1” is assigned to a first measuring device 10 as the device ID, and “D2” is assigned to a second measuring device 10 as the device ID. In the device identification information table, each device ID is associated with a supporter ID of a supporter to which this device ID is lent. For example, “S1” is associated with measuring device 10 of “D1” as the supporter ID, and “S2” is associated with measuring device 10 of “D2” as the supporter ID.

The model number, the serial number and the location of placement, of device identification information 524, are input to server device 50 by the service provider who provides the information providing service. Device identification information 524 may be input to server device 50 by an owner of measuring device 10. When the owner of measuring device 10 is a supporter, the supporter may input device identification information 524 using supporter terminal 30. Server device 50 stores obtained device identification information 524 in the device identification information table, thereby storing device identification information 524 in storage device 520.

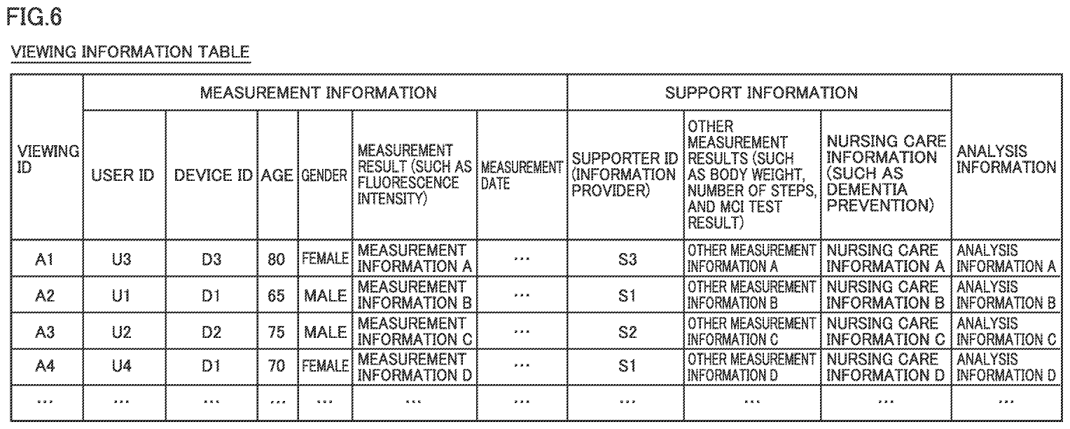

FIG. 6 is a diagram for illustrating a viewing information table stored in server device 50 according to the first embodiment. Server device 50 stores viewing information 525 using the viewing information table shown in FIG. 6.

As shown in FIG. 6, the viewing information table stores information about a user ID for identifying a user who is a subject of measurement of the AGEs, a device ID for identifying measuring device 10 used to measure the AGEs, an age of the user, a gender of the user, a measurement result of the AGEs such as fluorescence intensity, a measurement date and the like as the measurement information of viewing information 525.

The viewing information table stores information about a supporter ID for identifying a supporter who supports the user, other measurement results of the user other than the AGEs, nursing care information that is valuable for the user, and the like as the support information of viewing information 525.

Furthermore, the viewing information table stores information about a result obtained by analyzing the health condition of the user based on the measurement information and the support information, and the like as the analysis information of viewing information 525.

The measurement information, the support information and the analysis information as described above are linked to each other by a viewing ID. Server device 50 provides viewing information 525 to the user or the supporter using the viewing ID.

For example, when the user having the user ID “U1” requests viewing information 525 from server device 50 using user terminal 20, server device 50 outputs viewing information 525 corresponding to the viewing ID “A1” to user terminal 20. By looking at viewing information 525 displayed on user terminal 20, the user can obtain the measurement information, the support information and the analysis information corresponding to the viewing ID “A1”.

For example, when the supporter having the supporter ID “S1” requests viewing information 525 from server device 50 using supporter terminal 30, server device 50 outputs viewing information 525 corresponding to the viewing ID “A1” to supporter terminal 30. By looking at viewing information 525 displayed on supporter terminal 30, the supporter can obtain the measurement information, the support information and the analysis information corresponding to the viewing ID “A1”. Particularly, in the present embodiment, the supporter can obtain, from server device 50, the measurement information, the support information and the analysis information regarding the users associated with his/her own supporter ID, i.e., all of the users supported by the supporter himself/herself. For example, in the example shown in FIG. 6, the supporter having the supporter ID “S1” can obtain, from server device 50, the measurement information, the support information and the analysis information regarding each of the user having the user ID “U1” and the user having the user ID “U4”.

When the viewer specifies a user ID of a user whom the viewer wants to view and requests viewing information 525 from server device 50 using viewer terminal 40, server device 50 outputs viewing information 525 corresponding to the specified user ID to viewer terminal 40. By looking at viewing information 525 displayed on viewer terminal 40, the viewer can obtain the measurement information, the support information and the analysis information corresponding to the specified user ID.

### [Display Examples of Viewing Information]

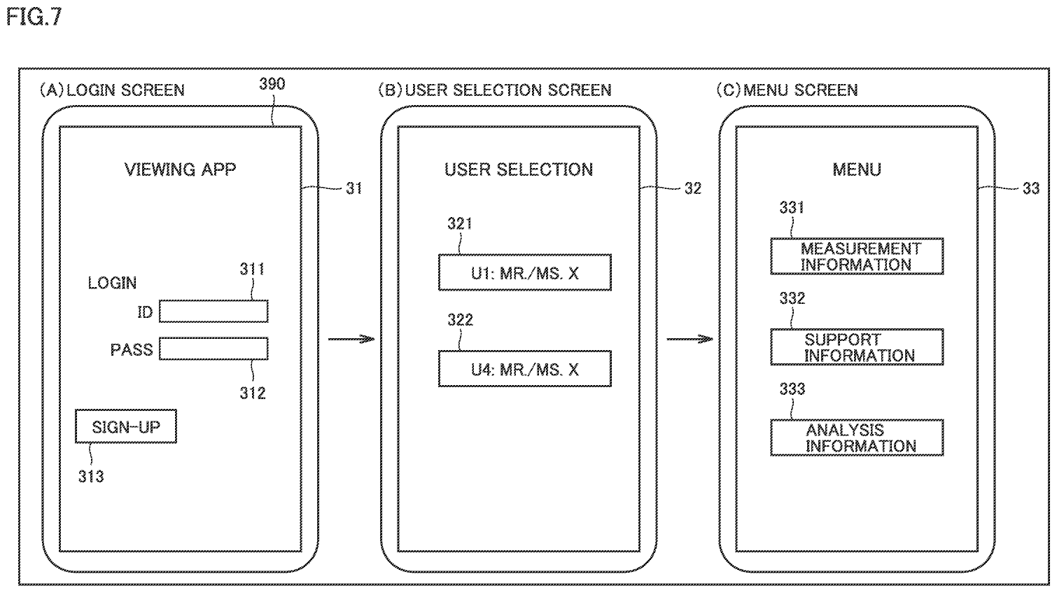

Display examples of the viewing information will be described with reference to FIGS. 7 to 13. Each of FIGS. 7 to 13 shows an example of a display screen in user terminal 20, supporter terminal 30 or viewer terminal 40 according to the first embodiment. Although each of FIGS. 7 to 13 shows the display example of the viewing information in supporter terminal 30, display examples of the viewing information in user terminal 20 and viewer terminal 40 are similar to the example shown in each of FIGS. 7 to 13.

When the supporter performs an application program for using the information providing service using supporter terminal 30, supporter terminal 30 causes a display 390 to display a login screen 31 shown in FIG. 7(A).

Login screen 31 includes an icon 311 for inputting the user ID, an icon 312 for inputting the password, and an icon 313 for making a sign-up.

When the supporter inputs the supporter ID and the password on login screen 31 shown in FIG. 7(A), supporter terminal 30 outputs the supporter ID and the password to server device 50. When server device 50 authenticates the supporter based on the supporter ID and the password, supporter terminal 30 causes display 390 to display a user selection screen 32 shown in FIG. 7(B).

User selection screen 32 is a screen for selecting a user about whom various types of information such as the measurement information are to be viewed. For example, in the case of the supporter having the supporter ID “S1”, icons 321 and 322 for selecting the user having the user ID “U1” and the user having the user ID “U4”, respectively, are displayed on display 390.

When the supporter selects the user on user selection screen 32 shown in FIG. 7(B), supporter terminal 30 causes display 390 to display a menu screen 33 shown in FIG. 7(C).

Menu screen 33 includes an icon 331 for viewing the measurement information of the viewing information, an icon 332 for viewing the support information of the viewing information, and an icon 333 for viewing the analysis information of the viewing information.

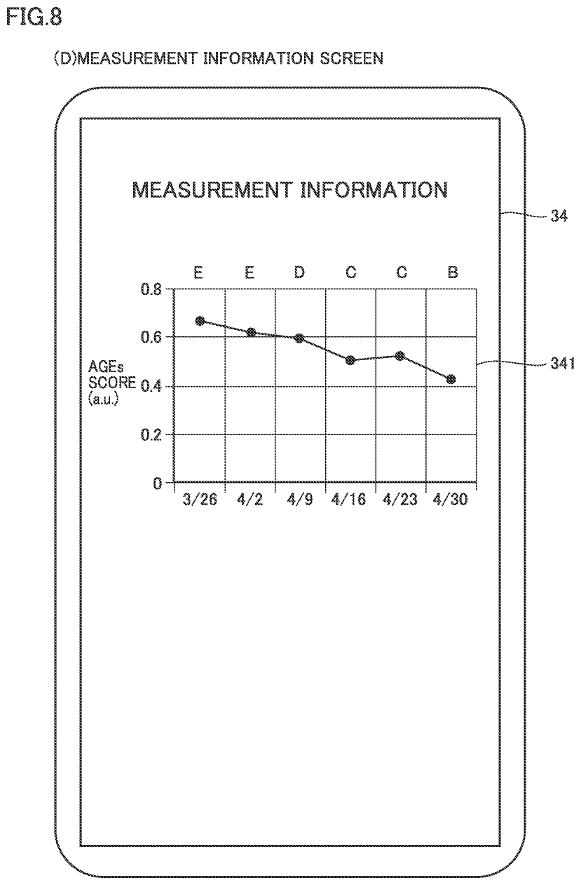

When the supporter clicks icon 331 on menu screen 33 shown in FIG. 7(C), supporter terminal 30 obtains the measurement information corresponding to the user associated with the supporter ID from server device 50. As shown in FIG. 8, supporter terminal 30 causes display 390 to display a measurement information screen 34 for viewing the obtained measurement information.

Measurement information screen 34 includes an image 341 showing time-lapse data of the AGEs measured periodically. In the example shown in FIG. 8, the measurement results of the AGEs measured every week are shown chronologically by a graph. By viewing such a screen, the supporter can view the time-lapse data of the AGEs about the desired user.

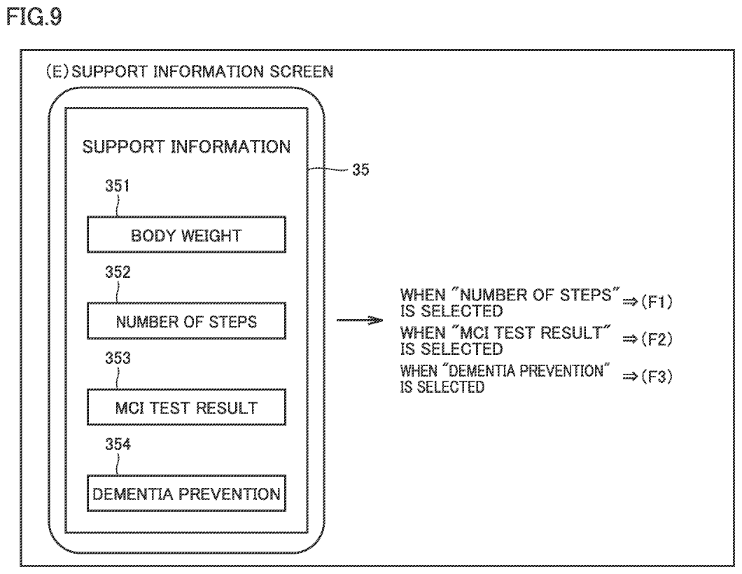

When the user clicks icon 332 on the menu screen shown in FIG. 7(C), supporter terminal 30 obtains the support information corresponding to the user from server device 50. As shown in FIG. 9(E), supporter terminal 30 causes display 390 to display a support information screen 35 for viewing the obtained support information.

As icons for viewing the other measurement results other than the AGEs, support information screen 35 includes an icon 351 for viewing the body weight of the user, an icon 352 for viewing the number of steps of the user, and an icon 353 for viewing the MCI test result of the user. Support information screen 35 further includes an icon 354 for viewing the advice about dementia prevention as an icon for viewing the nursing care information.

When the supporter clicks icon 351 on support information screen 35 shown in FIG. 9(E), supporter terminal 30 obtains data of the body weight measured in the past by the user from server device 50 and causes display 390 to display the obtained data of the body weight. For example, supporter terminal 30 causes display 390 to chronologically display the body weights measured a plurality of number of times in the past by the user.

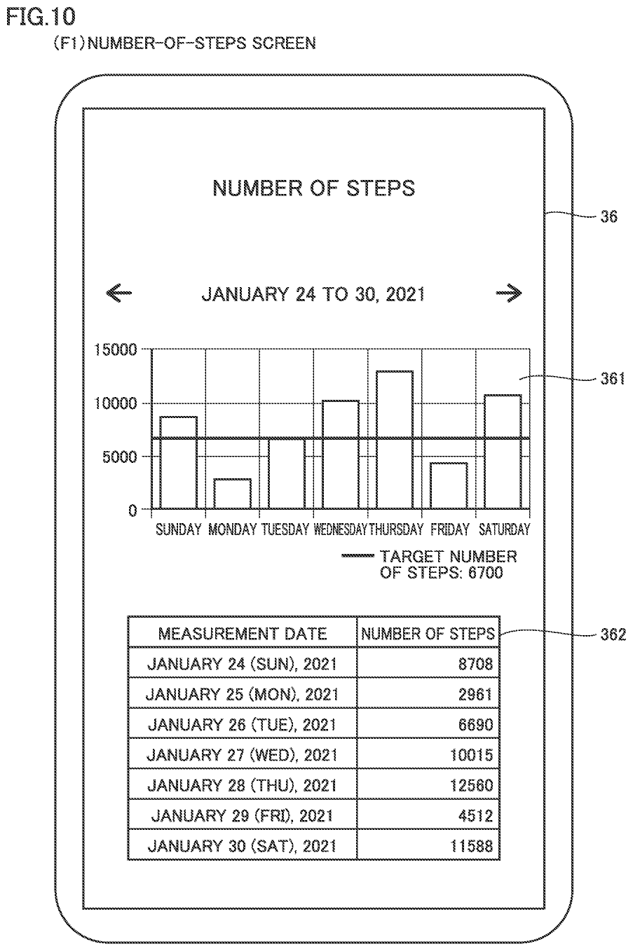

When the user clicks icon 352 on support information screen 35 shown in FIG. 9(E), supporter terminal 30 obtains the number of steps corresponding to the user from server device 50. As shown in FIG. 10 (F1), supporter terminal 30 causes display 390 to display a number-of-steps screen 36 for viewing the obtained number of steps.

Number-of-steps screen 36 includes an image 361 including a graph that chronologically shows the number of steps in one day measured a plurality of number of times in the past by the user, and an image 362 including a table that chronologically shows the number of steps in one day measured a plurality of number of times in the past by the user. Thus, the supporter can view the number of steps in one day measured in the past by the user.

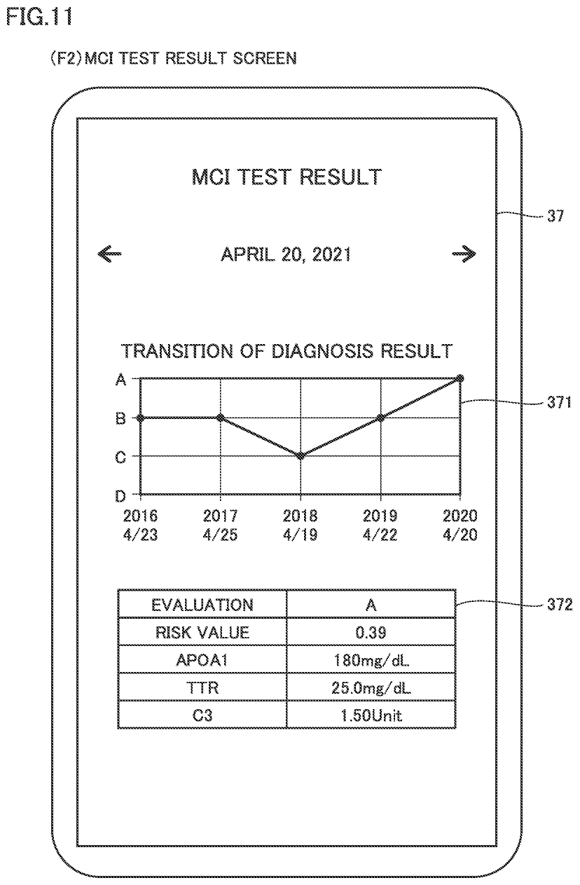

When the user clicks icon 353 on support information screen 35 shown in FIG. 9(E), supporter terminal 30 obtains the MCI test result corresponding to the user from server device 50. As shown in FIG. 11 (F2), supporter terminal 30 causes display 390 to display an MCI test result screen 37 for viewing the obtained MCI test result.

MCI test result screen 37 includes an image 371 including a graph that chronologically shows the MCI test results obtained a plurality of number of times in the past by the user, and an image 372 including a table that shows the MCI test result obtained in the past by the user. Thus, the supporter can view the MCI test result obtained in the past by the user.

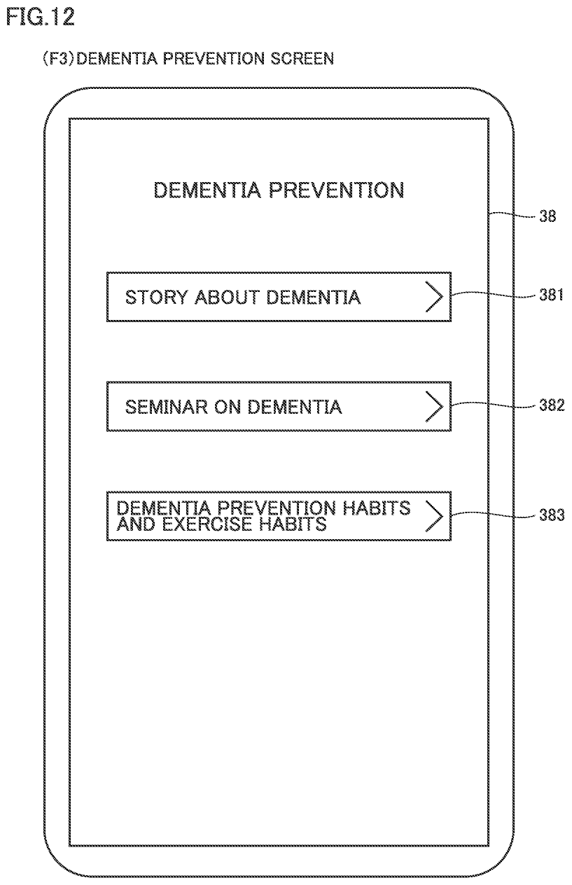

When the user clicks icon 354 on support information screen 35 shown in FIG. 9(E), supporter terminal 30 obtains the advice about dementia prevention corresponding to the user from server device 50. As shown in FIG. 12 (F3), supporter terminal 30 causes display 390 to display a dementia prevention screen 38 for viewing the obtained advice about dementia prevention.

Dementia prevention screen 38 includes an icon 381 for viewing a description about dementia, an icon 382 for viewing details of a seminar on dementia, and an icon 383 for viewing details about dementia prevention habits and exercise habits. By clicking these icons, the supporter can view various advices about dementia prevention provided to the user in the past.

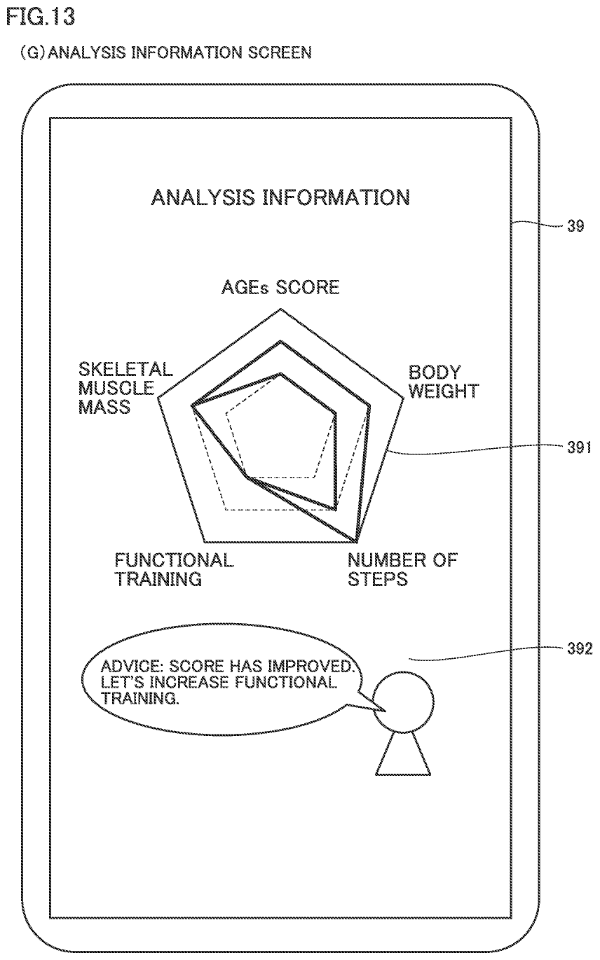

When the user clicks icon 333 on menu screen 33 shown in FIG. 7(C), supporter terminal 30 obtains the analysis information corresponding to the user from server device 50. As shown in FIG. 13 (G), supporter terminal 30 causes display 390 to display an analysis information screen 39 for viewing the obtained analysis information.

Analysis information screen 39 includes an image 391 showing a result obtained by analyzing the health condition of the user based on the value of the degree of accumulation of the AGEs converted into a score, which is included in the measurement information, and the body weight, the number of steps, the skeletal muscle mass, and the functional training result of the user, which are included in the support information, and an image 392 showing an advice for the user obtained based on the analysis result. Thus, the supporter can view the analysis result of the health condition of the user and the advice.

### [Process of Information Processing System]

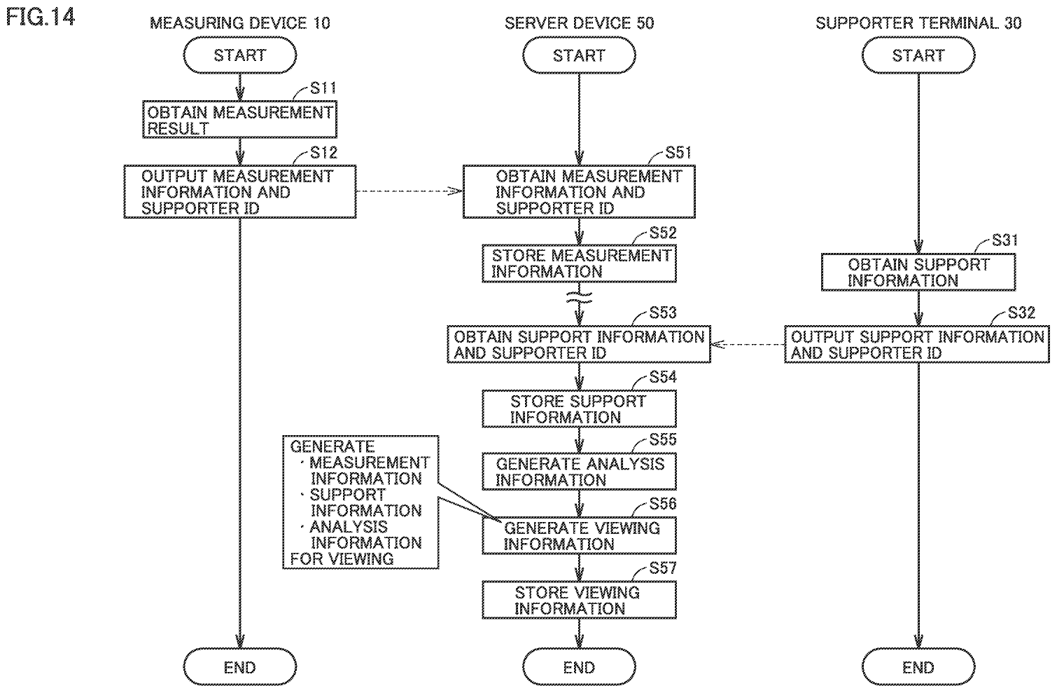

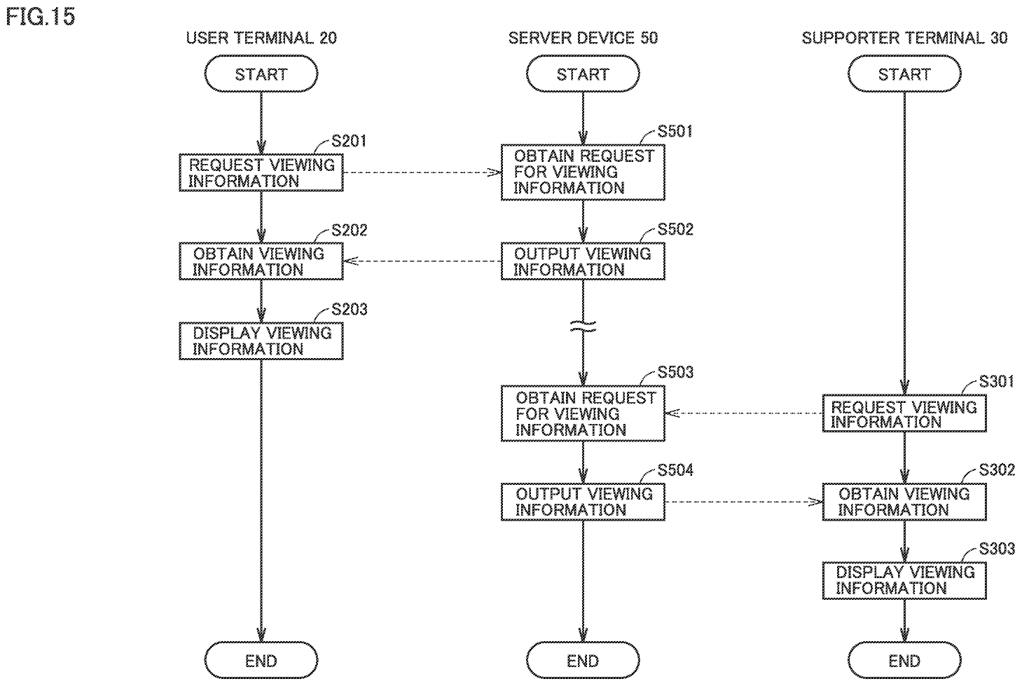

A process of information processing system 1 will be described with reference to FIGS. 14 and 15. FIG. 14 is a flowchart of a storage process performed by the information processing system according to the first embodiment. Of the process steps (hereinafter, abbreviated as “S”) shown in FIGS. 14 and 15, a process performed by server device 50 is implemented by controller 510 performing information processing program 521.

FIG. 14 is a flowchart of the storage process performed by information processing system 1 according to the first embodiment. As shown in FIG. 14, when measuring device 10 measures AGEs of a user and obtains measurement information including a measurement result of the AGEs (S11), measuring device 10 outputs the obtained measurement information and identification information (supporter ID) of a supporter assigned to measuring device 10 to server device 50 (S12). At this time, measuring device 10 outputs the measurement information in association with the supporter ID to server device 50. For example, the user inputs a user ID, an age, a gender and the like on the input screen displayed on the display of measuring device 10, and measures the AGEs using measuring device 10. Measuring device 10 outputs the user ID, the age and the gender input by the user and the measurement result of the user in association with the supporter ID to server device 50 as the measurement information.

When server device 50 obtains the measurement information and the supporter ID from measuring device 10 (S51), server device 50 stores the obtained measurement information in storage device 520 in association with the user ID and the supporter ID (S52).

When supporter terminal 30 obtains support information (S31), supporter terminal 30 outputs the obtained support information and the supporter ID to server device 50 (S32). At this time, supporter terminal 30 outputs the measurement information in association with the supporter ID to server device 50. For example, while viewing the measurement information of the user, the supporter inputs the supporter ID, other measurement results of the user other than the AGEs, and nursing care information on the input screen displayed on the display of supporter terminal 30. Supporter terminal 30 outputs the other measurement results and the nursing care information in association with the supporter ID to server device 50 as the support information.

When server device 50 obtains the support information and the supporter ID from supporter terminal 30 (S53), server device 50 stores the obtained support information in storage device 520 in association with the user ID and the supporter ID (S54).

Server device 50 analyzes a health condition of the user based on the measurement information and the support information, and generates analysis information including an analysis result (S55). Server device 50 generates viewing information based on the measurement information obtained from measuring device 10, the support information obtained from supporter terminal 30, and the analysis information (S56). At this time, server device 50 creates time-lapse data of the AGEs for each user as the viewing information, based on the measurement result of the AGEs of each user transmitted from measuring device 10 periodically. Server device 50 stores the generated viewing information using the viewing information table shown in FIG. 6 (S57).

Information processing system 1 repeatedly performs the process shown in FIG. 14, and thereby, server device 50 can accumulate and store the viewing information including the measurement information obtained from measuring device 10, the support information obtained from supporter terminal 30, and the analysis information.

FIG. 15 is a flowchart of a viewing process performed by information processing system 1 according to the first embodiment. As shown in FIG. 15, user terminal 20 requests the viewing information from server device 50 based on user's operation (S201). At this time, user terminal 20 outputs, to server device 50, the user ID input at login.

For example, the user can request the measurement information from server device 50 by clicking icon 331 on menu screen 33 shown in FIG. 7. The user can request the support information from server device 50 by clicking icon 332 on menu screen 33 shown in FIG. 7. The user can request the analysis information from server device 50 by clicking icon 333 on menu screen 33 shown in FIG. 7.

When server device 50 obtains the request for the viewing information and the user ID from user terminal 20 (S501), server device 50 identifies the viewing information corresponding to the user based on the user ID of the user who is a requestor, and outputs the identified viewing information to user terminal 20 (S502).

When user terminal 20 obtains the viewing information from user terminal 20 (S202), user terminal 20 causes the display to display the obtained viewing information (S203). As a result, the user can view the viewing information accumulated and stored in server device 50, using user terminal 20.

Supporter terminal 30 requests the viewing information from server device 50 based on supporter's operation (S301). At this time, supporter terminal 30 outputs, to server device 50, the supporter ID input at login.

For example, the supporter can request the measurement information from server device 50 by clicking icon 331 on menu screen 33 shown in FIG. 7. The supporter can request the support information from server device 50 by clicking icon 332 on menu screen 33 shown in FIG. 7. The supporter can request the analysis information from server device 50 by clicking icon 333 on menu screen 33 shown in FIG. 7.

When server device 50 obtains the request for the viewing information and the supporter ID from supporter terminal 30 (S503), server device 50 identifies the viewing information corresponding to the user supported by the supporter based on the supporter ID, and outputs the identified viewing information to supporter terminal 30 (S504).

When supporter terminal 30 obtains the viewing information from server device 50 (S302), supporter terminal 30 causes display 390 to display the obtained viewing information (S303). As a result, the supporter can view the viewing information accumulated and stored in server device 50, using supporter terminal 30.

Although the viewing process performed by user terminal 20 and supporter terminal 30 has been described in the example shown in FIG. 15 above, viewer terminal 40 also performs a process similar to the viewing process performed by user terminal 20 and supporter terminal 30.

As described above, in information processing system 1 according to the first embodiment, the user, the supporter and the viewer can conveniently share the measurement result of the AGEs.

### [Modification of Information Processing System According to First Embodiment]

Although information processing system 1 according to the first embodiment has been described above, various modifications and applications are possible in information processing system 1 according to the first embodiment. Only a difference between a modification of information processing system 1 according to the first embodiment and information processing system 1 according to the first embodiment will be described below.

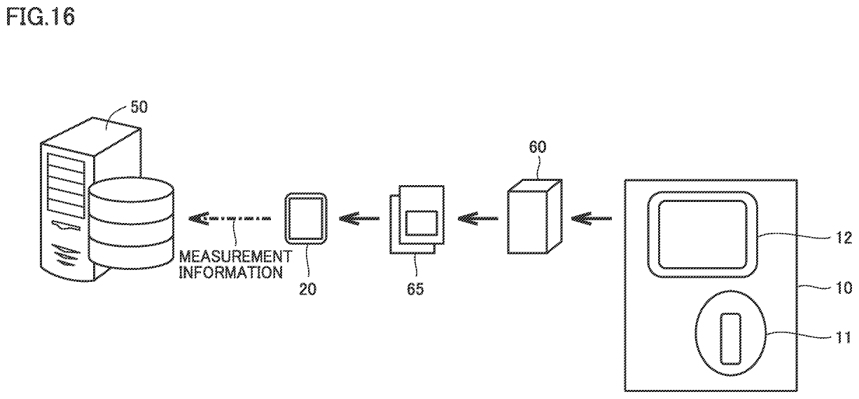

FIG. 16 shows the modification of information processing system 1 according to the first embodiment. As shown in FIG. 16, the information processing system according to the modification further includes a printer 60 communicably connected to measuring device 10.

When measuring device 10 measures AGEs of a user, measuring device 10 generates a one-dimensional or two-dimensional code based on measurement information including a measurement result of the AGEs, and outputs information about the code to printer 60. Printer 60 prints the code including the measurement information onto a receipt 65. The user reads the code printed on receipt 65, using a camera of user terminal 20, to thereby obtain the measurement information, and outputs the obtained measurement information to server device 50. Server device 50 stores, as viewing information, the measurement information obtained from user terminal 20 in association with the user ID and the supporter ID.

Not only user terminal 20 but also supporter terminal 30 may read the code printed on receipt 65, using a camera, to thereby obtain the measurement information, and output the obtained measurement information to server device 50. Server device 50 may store, as viewing information, the measurement information obtained from supporter terminal 30 in association with the user ID and the supporter ID.

As described above, in the information processing system according to the modification, server device 50 may not only obtain the measurement information directly from measuring device 10, but also obtain the measurement information through user terminal 20 or supporter terminal 30.

### <Information Processing System According to Second Embodiment>

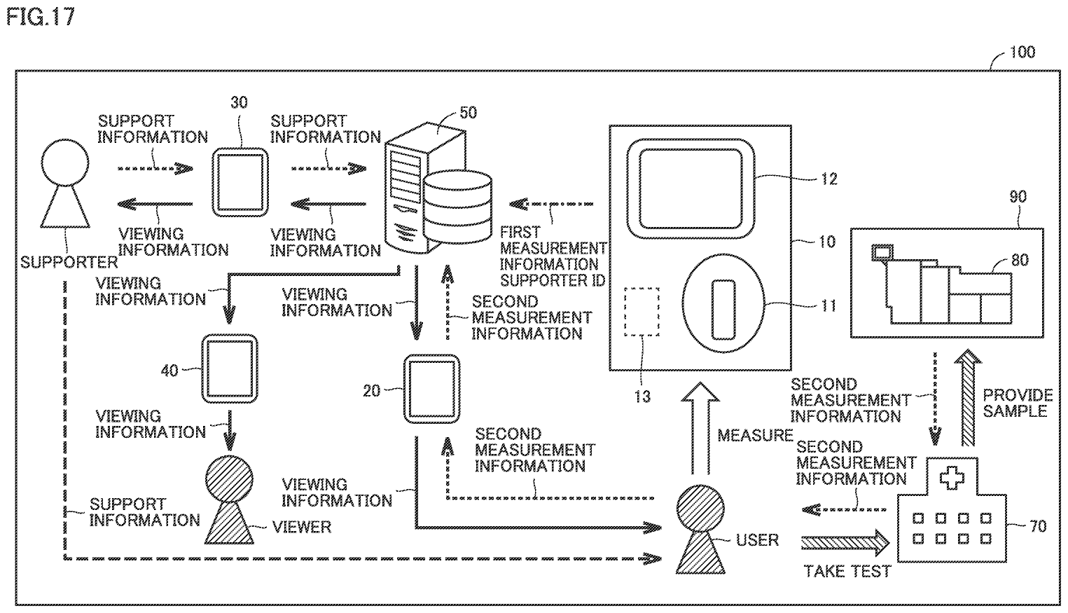

Only a difference between an information processing system 100 according to a second embodiment and information processing system 1 according to the first embodiment will be described below. FIG. 17 shows information processing system 100 according to the second embodiment.

As shown in FIG. 17, information processing system 100 according to the second embodiment includes a measuring device 80 that measures AGEs based on a sample obtained from a user, in addition to measuring device 10 that measures the AGEs of the user in a non-invasive manner. In FIG. 17, measurement information provided from measuring device 10 to server device 50 will be referred to as “first measurement information” and measurement information provided from measuring device 80 to server device 50 will be referred to as “second measurement information”.

As described above, measuring device 10 measures the AGEs by measuring the intensity of fluorescence generated when applying excitation light to the skin of the user. In contrast, measuring device 80 measures the AGEs by analyzing the sample obtained from the user, and can measure the AGEs in more detail than measuring device 10. Examples of the sample include blood, plasma, serum, hair, nail, and urine, and serum is preferable from the perspective of ease of analysis. Examples of measuring device 80 include liquid chromatography/mass spectrometry (LC-MS/MS), which is a combination of high performance liquid chromatography and a triple quadrupole mass spectrometer.

Specifically, the user takes a test in a medical institution 70. Medical institution 70 obtains a sample of the user and provides the obtained sample to an analytical business entity 90. Analytical business entity 90 analyzes the sample of the user using measuring device 80, to thereby obtain a measurement result of the AGEs. Measuring device 80 measures the AGEs for each of a plurality of compounds that can be detected from the sample.

Analytical business entity 90 provides second measurement information including the measurement result obtained by measuring device 80 to medical institution 70. Medical institution 70 provides the second measurement information obtained from analytical business entity 90 to the user. For example, as described in the example shown in FIG. 16, medical institution 70 provides the second measurement information to the user by printing a code including the second measurement information onto receipt 65. Similarly to the first measurement information, the second measurement information includes a user ID, a device ID of measuring device 80, an age, a gender, a measurement result, a measurement date and the like as shown in FIG. 6.

The user reads the code printed on receipt 65, using a camera of user terminal 20, to thereby obtain the second measurement information, and outputs the obtained second measurement information to server device 50. Server device 50 stores the second measurement information obtained from user terminal 20 in association with the user ID and the supporter ID.

Not only user terminal 20 but also supporter terminal 30 may read the code printed on receipt 65, using a camera, to thereby obtain the second measurement information, and output the obtained second measurement information to server device 50. Server device 50 may store the second measurement information obtained from supporter terminal 30 in association with the user ID and the supporter ID.

As described above, server device 50 obtains and stores the measurement result of the AGEs (first measurement information) obtained as a result of non-invasive measurement by measuring device 10, and obtains and stores the measurement result of the AGEs (second measurement information) obtained as a result of measurement using the sample of the user by measuring device 80. Server device 50 stores at least one of the first measurement information and the second measurement information as viewing information. Furthermore, server device 50 analyzes a health condition of the user based on at least one of the first measurement information and the second measurement information and the support information obtained from supporter terminal 30, and stores analysis information including an analysis result as viewing information. As described above, in information processing system 100 according to the second embodiment, the user and the supporter can conveniently share not only the measurement result obtained as a result of non-invasive measurement by measuring device 10 but also the measurement result obtained as a result of non-invasive measurement by measuring device 80.

### [Modification of Information Processing System According to Second Embodiment]

Although information processing system 100 according to the second embodiment has been described above, various modifications and applications are possible in information processing system 100 according to the second embodiment. Only a difference between a modification of information processing system 100 according to the second embodiment and information processing system 100 according to the second embodiment will be described below.

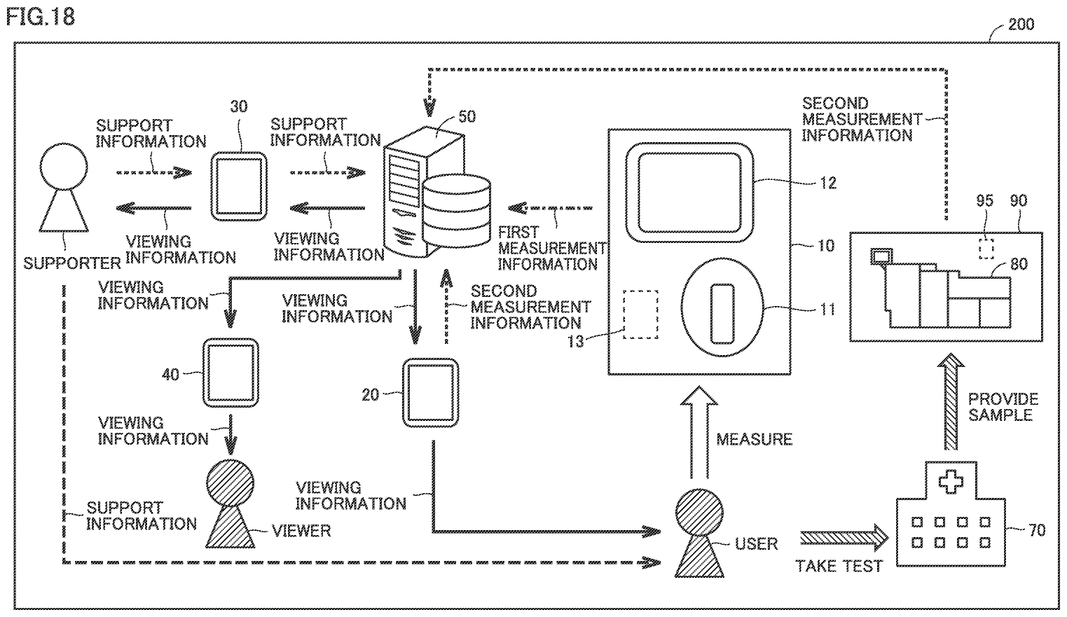

FIG. 18 shows the modification of information processing system 100 according to the second embodiment. As shown in FIG. 18, in an information processing system 200 according to the modification, analytical business entity 90 directly provides the second measurement information to server device 50.

Specifically, analytical business entity 90 includes a communication unit 93. Communication unit 93 receives and transmits data (information) to and from server device 50 by wired communication or wireless communication. Communication unit 93 is an information terminal that can communicate with server device 50 through the network, such as a desktop PC, a laptop PC, a smartphone, a smart watch, a wearable device, a tablet PC, and a network adapter. Communication unit 93 may be integrated with measuring device 80.

Analytical business entity 90 outputs the second measurement information including the measurement result obtained by measuring device 80 to server device 50 using communication unit 93. Server device 50 stores the second measurement information obtained from communication unit 93 of analytical business entity 90.

As described above, in information processing system 200 according to the modification, server device 50 may not only obtain the measurement information obtained by measuring device 80 through user terminal 20, but also obtain the measurement information obtained by measuring device 80 directly from analytical business entity 90.

Although the information processing system according to the second embodiment has been described above, server device 50 may store only the first measurement information obtained by measuring device 10 as viewing information, or may store only the second measurement information obtained by measuring device 80 as viewing information, or may store both the first measurement information and the second measurement information as viewing information.

### Aspects

It will be understood by those skilled in the art that the plurality of exemplary embodiments described above are specific examples of the following aspects.

(Clause 1) An information processing system according to an aspect includes: a measuring device that measures advanced glycation end products (AGEs) of a user and is provided with identification information of a supporter who supports the user; and a server device that communicates with the measuring device and a supporter terminal of the supporter. The measuring device outputs a measurement result of the AGEs in association with the identification information to the server device. The server device stores the measurement result in association with the identification information and outputs information about the measurement result to the supporter terminal.

According to the information processing system described in Clause 1, each of the user and the supporter can obtain the measurement information from server device 50. Thus, the user and the supporter can conveniently share the measurement result of the AGEs. Furthermore, the supporter can obtain the measurement result associated with his/her own identification information (supporter ID).

(Clause 2) The server device outputs the information about the measurement result to the supporter terminal in response to a request from the supporter terminal.

According to the information processing system described in Clause 2, the supporter can obtain the measurement result by requesting the measurement result from server device 50 using supporter terminal 30.

(Clause 3) The server device communicates with at least one of a user terminal of the user and a viewer terminal of a viewer authorized to view the measurement result, and outputs the information about the measurement result to at least one of the user terminal and the viewer terminal.

According to the information processing system described in Clause 3, the user can obtain the measurement result from server device 50 using user terminal 20. Furthermore, the viewer can obtain the measurement result from server device 50 using viewer terminal 40.

(Clause 4) The server device obtains support information for supporting the user from the supporter terminal and stores the support information, and outputs the support information to at least one of the user terminal, the viewer terminal and the supporter terminal.

According to the information processing system described in Clause 4, each of the user, the viewer and the supporter can obtain the support information from server device 50. Thus, the user, the viewer and the supporter can conveniently share the support information provided by the supporter.

(Clause 5) The server device analyzes a health condition of the user based on the measurement result and the support information, and stores an analysis result, and outputs information about the analysis result to at least one of the user terminal, the viewer terminal and the supporter terminal.

According to the information processing system described in Clause 5, each of the user, the viewer and the supporter can obtain the analysis result of the health condition of the user from server device 50. Thus, the user, the viewer and the supporter can conveniently share the analysis result.

(Clause 6) The support information includes information about lifestyle-related diseases prevention of the user.

According to the information processing system described in Clause 6, the user and the supporter can conveniently share the information about lifestyle-related diseases prevention of the user.

(Clause 7) The measuring device measures the AGEs of the user in a non-invasive manner.

According to the information processing system described in Clause 7, the user and the supporter can conveniently share the measurement result obtained by measuring device 10 that measures the AGEs in a non-invasive manner.

(Clause 8) The measuring device measures the AGEs based on a sample obtained from the user.

According to the information processing system described in Clause 8, the user and the supporter can conveniently share the measurement result obtained by measuring device 80 that measures the AGEs based on the sample obtained from the user.

(Clause 9) An information processing method according to an aspect includes: (a) outputting, by a measuring device, to a server device, a measurement result of AGEs of a user in association with identification information of a supporter who supports the user; (b) storing, by the server device, the measurement result in association with the identification information; and (c) outputting, by the server device, information about the measurement result to a supporter terminal of the supporter.

According to the information processing method described in Clause 9, each of the user and the supporter can obtain the measurement information from the server device. Thus, the user and the supporter can conveniently share the measurement result of the AGEs. Furthermore, the supporter can obtain the measurement result associated with his/her own identification information (supporter ID).

### REFERENCE SIGNS LIST

1, 100, 200 information processing system; 10, 80 measuring device; 11 measuring unit; 12, 390 display; 13, 93 communication unit; 20 user terminal; 30 supporter terminal; 31 login screen; 32 user selection screen; 33 menu screen; 34 measurement information screen; 35 support information screen; 36 number-of-steps screen; 37 test result screen; 38 dementia prevention screen; 39 analysis information screen; 40 viewer terminal; 50 server device; 60 printer; 65 receipt; 70 medical institution; 90 analytical business entity; 311, 312, 313, 321, 322, 331, 332, 333, 351, 352, 353, 354, 381, 382, 383 icon; 341, 361, 362, 371, 372, 391, 392 image; 510 controller, 520 storage device; 521 information processing program; 522 user identification information; 523 supporter identification information; 524 device identification information; 525 viewing information; 530 communication device.

1. An information processing system comprising:
a measuring device that measures advanced glycation end products (AGEs) of a user and is provided with identification information of a supporter who is supposed to give a healthcare support to the user; and
a server device that communicates with the measuring device and a supporter terminal utilized by the supporter, wherein
the measuring device outputs a measurement result of the AGEs for the user in association with the identification information to the server device, and
the server device generates information for viewing for the user based on the measurement result, identifies the supporter based on the identification information, and output the information for viewing to the supporter terminal.

2. The information processing system according to claim 1, wherein
the server device outputs the information for viewing to the supporter terminal in response to a request from the supporter terminal.

3. The information processing system according to claim 1, wherein
the server device
communicates with at least one of a user terminal of the user and a viewer terminal of a viewer authorized to view the measurement result, and
outputs the information for viewing to at least one of the user terminal and the viewer terminal.

4. The information processing system according to claim 3, wherein
the server device
obtains support information for supporting the user from the supporter terminal and stores the support information, and
outputs the support information to at least one of the user terminal, the viewer terminal and the supporter terminal.

5. The information processing system according to claim 4, wherein
the server device
analyzes a health condition of the user based on the measurement result and the support information, and stores an analysis result, and
outputs information about the analysis result to at least one of the user terminal, the viewer terminal and the supporter terminal.

6. The information processing system according to claim 4, wherein
the support information includes information about lifestyle-related diseases prevention of the user.

7. The information processing system according to claim 1, wherein
the measuring device measures the AGEs of the user in a non-invasive manner.

8. The information processing system according to claim 1, wherein
the measuring device measures the AGEs based on a sample obtained from the user.

9. An information processing method comprising:
measuring, by a measuring device, advanced glycation end products (AGEs) of a user,
outputting, by a measuring device, to a server device, a measurement result of the AGEs for the user in association with identification information of a supporter who is supposed to give a healthcare support to the user;
storing, by the server device, the measurement result in association with the identification information;
generating, by the server device, information for viewing for the user based on the measurement result to identify the supporter based on the identification information and
outputting, by the server device, the information for viewing to a supporter terminal of the supporter.
